(19) United States
(12) Patent Application Publication
(10) Pub. No.: US 2006/0210546 A1
(43) Pub. Date: Sep. 21, 2006

(54) Athlete dietary supplement

(75) Inventors: Brian V. Sokol, Vermilion, OH (US); Scott Short, Avon Lake, OH (US)

Correspondence Address: Fay, Sharpe, Fagan, Minnich & McKee, LLP;7th Floor, 1100 Superior Avenue, Cleveland, OH, 44114-2579, US

(73) Assignee: Activity Targeted Supplements, LLC

(21) Appl. No.: 11/377,500

(22) Filed: Mar. 16, 2006

Related U.S. Application Data
(60) Provisional application No. 60/663,129, filed on Mar. 18, 2005.

(51) Int. Cl. A61K 38/43; A61K 31/714; A61K 31/525; A61K 31/51; A61K 31/4415; A61K 31/455; A61K 31/473; A61K 33/06; A61K 31/045; A61K 36/76; A61K 36/16

(52) U.S. Cl. 424/94.1; 424/641; 424/771; 424/682; 424/752; 424/722; 514/52; 514/251; 514/276; 514/350; 514/356; 514/295; 514/729; 514/567; 514/553

(57) ABSTRACT

A vitamin supplement to temporarily enhance the abilities of a individual during a sport activity which includes a plurality of B family vitamins and one or more other vitamins, minerals, and/or natural ingredients.

The present invention claims priority on Application Ser. No. 60/663,129 filed Mar. 18, 2005, which is incorporated herein by reference.

The present invention is directed to vitamin supplements and more particularly to a vitamin supplement for athletes, and still more particularly to a vitamin supplement for golfers.

### BACKGROUND OF THE INVENTION

The sports nutrition industry encompasses a wide range of products, including capsules, tablets, powders, liquid beverages, gelatins, food bars, and beverage mixes. These products are marketed to individuals as aids and/or supplements which will facilitate in boasting and/or maintaining the performance of an individual when performing one or more physical activities. Many of these products are marketed and directed to certain types of sport activities.

Many prior vitamin supplements required a user to ingest the vitamin supplement for an extended period of time, typically 14-30 days, so as to build up certain vitamin levels in the body to achieve the asserted effect of the vitamin supplement. As such, the user must consume multiple vitamin supplements over an extended period of time before the asserted effect of the vitamin supplement can be realized by the user. In addition to the inconvenience and expense associated with the taking of the vitamin supplement on a daily basis for an extended period of time, the user is subjected to the effects of the vitamin supplement over long periods of time. As such, the user may want the effects of the vitamin supplement for one type of activity, but may not want the effects of the vitamin supplements for other types of activities.

In view of the current state of the art associated with vitamin supplements, there is a need for a vitamin supplement that can be taken the same day as the anticipated activity of the user to provide the user with a quick and effective dosage of vitamins to enhance the ability of the user for a certain activity over at least a several hour period of time.

### SUMMARY OF THE INVENTION

The present invention is directed to vitamin supplements, more particularly to a vitamin supplement for athletes, still more particularly to a vitamin supplement for athletes that can be taken the same day as an athletic event and quickly and effectively provide effective vitamin levels to the athlete to enhance the ability of the athlete.

In one non-limiting aspect of the present invention, there is provided a vitamin supplement that can be used to enhance one or more abilities of an athlete in various types of sporting activities such as, but not limited to, archery, baseball, basketball, bicycling, bobsledding, bowling, boxing, car racing, canoeing, cricket, curling, football, golfing, gymnastics, ice hockey, horse riding, ice skating, kayaking, luge, motorcycle racing, motorboat racing, pool, roller skating, racquetball, roller blading, rugby, running, sailing, skeleton, skateboarding, ski jumping, snow boarding, snow skiing, soccer, surfing, swimming, table tennis, target shooting, tennis, water skiing, weight lifting, etc. The vitamin supplement can be used by professional athletes, weekend athlete and/or the average person engaging in a particular activity on a particular day. Although the vitamin supplement of the present invention is particularly directed to enhance one or more abilities of a user while the user engages in one or more sport activities; it will be appreciated that the vitamin supplement of the present invention can be used to enhance one or more ability of the individual in act ivies other than sporting activities. For example, a laborer may take the vitamin supplement to enhance the laborer's abilities during the job. In another example, a business person may take the vitamin supplement of the present invention before an important meeting and/or presentation to help ensure that the business person is alert and/or energetic during such meeting and/or presentation. As such, the vitamin supplement of the present invention can be used to temporarily enhance one or more of the user's abilities during day to day activities. In one non-limiting embodiment of the invention, the vitamin supplement of the present invention includes all natural ingredients. In another and/or alternative non-limiting embodiment of the present invention, the vitamin supplement of the present invention is formulated to reduce pain, reduce anxiety and/or improve the focus of the user during an activity. As such, the vitamin supplement can improve the outlook a user feels, improve the mental attitude of the user, and/or reduce the anxiety levels and/or improve the focus of a user, and/or improve actual and/or perceived energy and/or endurance levels of the user (e.g., positively affects the mind and body in the areas of mental focus, hand/eye coordination, depth perception, flexibility, stamina and/or energy; reduce fatigue, muscle pain, anxiety and/or stress. etc.), thereby facilitating in improving the performance of the user during a certain sport and/or task. The vitamin supplement of the present invention thus provides a dosage of vitamins and minerals which can enhance the body's ability to perform a physical activity, without the use of caffeine, sugars, or calories, and without sedating the user. In still another and/or alternative embodiment of the present invention, the vitamin supplement of the present invention enables a user to take a dosage of the vitamin supplement the same day of the activity by the user and still achieve the desired results from the vitamin supplement. As a result, there is no need for multiple day prior dosages (e.g., 30 days prior dosage) of the vitamin supplement to build up ingredients in the body of the user, thereby overcoming past limitations of prior vitamin supplements.

In another and/or alternative non-limiting aspect of the present invention, the vitamin supplement of the present invention includes a novel formulation of vitamin B family ingredients. The vitamin B ingredients include a plurality of Vitamin B ingredients that includes Vitamin B1, Vitamin B2, Vitamin B3, Vitamin B5, Vitamin B6, and/or Vitamin B12. In one non-limiting embodiment of the invention, the vitamin supplement includes at least three different types of Vitamin B. In another and/or alternative non-limiting embodiment of the invention, the vitamin supplement includes at least five different types of Vitamin B. In another and/or alternative embodiment of the invention, the vitamin supplement includes six different types of Vitamin B. In still another and/or alternative embodiment of the invention, the amount of vitamin B ingredients in the vitamin supplement is at least about 350 mg. In one non-limiting aspect of this embodiment, the amount of vitamin B ingredients in the vitamin supplement is at least about 375 mg. In another and/or alternative non-limiting aspect of this embodiment, the amount of vitamin B ingredients in the vitamin supplement is at least about 400 mg. In still another and/or alternative non-limiting aspect of this embodiment, the amount of vitamin B ingredients in the vitamin supplement is more than 400 mg. In yet another and/or alternative non-limiting aspect of this embodiment, the commutative amount of vitamin B1, vitamin B2, vitamin B5, and vitamin B6 in the vitamin supplement is at least about 300 mg. In still yet another and/or alternative non-limiting aspect of this embodiment, the commutative amount of vitamin B1, vitamin B2, vitamin B5, and vitamin B6 in the vitamin supplement is at least about 325 mg. In another and/or alternative non-limiting aspect of this embodiment, the commutative amount of vitamin B1, vitamin B2, vitamin B5, and vitamin B6 in the vitamin supplement is at least about 350 mg. In still another and/or alternative non-limiting aspect of this embodiment, the commutative amount of vitamin B1, vitamin B2, vitamin B5, and vitamin B6 in the vitamin supplement is at least about 375 mg. In yet another and/or alternative non-limiting aspect of this embodiment, the commutative amount of vitamin B1, vitamin B2, vitamin B5, and vitamin B6 in the vitamin supplement is at least about 400 mg. In still another and/or alternative non-limiting aspect of this embodiment, the amount of vitamin B1, vitamin B2, vitamin B5, and vitamin B6 in the vitamin supplement is about the same. In still yet another and/or alternative non-limiting aspect of this embodiment, the amount of vitamin B3 in the vitamin supplement is at least about 2 mg. In another and/or alternative non-limiting aspect of this embodiment, the amount of vitamin B3 in the vitamin supplement is about 2-50 mg. In still another and/or alternative non-limiting aspect of this embodiment, the amount of vitamin B3 in the vitamin supplement is about 5-25 mg. In yet another and/or alternative non-limiting aspect of this embodiment, the amount of vitamin B3 in the vitamin supplement is about 5-15 mg. In still yet another and/or alternative non-limiting aspect of this embodiment, the amount of vitamin B12 in the vitamin supplement is at least about 200 mcg. In another and/or alternative non-limiting aspect of this embodiment, the amount of vitamin B12 in the vitamin supplement is less than about 2000 mcg. In still another and/or alternative non-limiting aspect of this embodiment, the amount of vitamin B12 in the vitamin supplement is about 400-1800 mcg. In yet another and/or alternative non-limiting aspect of this embodiment, the amount of vitamin B12 in the vitamin supplement is about 500-1600 mcg. In still yet another and/or alternative non-limiting aspect of this embodiment, the amount of vitamin B12 in the vitamin supplement is about 800-1500 mcg. In another and/or alternative non-limiting aspect of this embodiment, the amount of vitamin B12 in the vitamin supplement is about 1000-1400 mcg. A general formulation and several more specific vitamin B ingredients of the vitamin supplement of the present invention are set forth as follows:

GENERAL FORMULATION

| | |
|---|---|
| Vitamin B Ingredients | at least 350 mg |
| (B1, B2, B3, B5, B6 and/or B12) | |

SPECIFIC FORMULATIONS

| | Example/Ingredient | | | | | |
|---|---|---|---|---|---|---|
| | Ex. 1 | | Ex. 2 | | Ex. 3 | |
| B1 | 25-300 | mg | 50-200 | mg | 75-150 | mg |
| B2 | 25-300 | mg | 50-200 | mg | 75-150 | mg |
| B3 | 1-50 | mg | 5-30 | mg | 5-15 | mg |
| B5 | 25-300 | mg | 50-200 | mg | 75-150 | mg |
| B6 | 25-300 | mg | 50-200 | mg | 75-150 | mg |
| B12 | 100-2000 | mcg | 500-1500 | mcg | 800-1400 | mcg |

The amount of the vitamin B ingredients in the vitamin supplement of the present invention can be tailored for 1) a particular sport and/or activity, 2) a particular sex, and/or 3) a body weight range; however, this is not required. As can be appreciated, the amount of the vitamin B ingredients in the vitamin supplement of the present invention can be tailored by other or additional factors.

In still another and/or alternative non-limiting aspect of the present invention, the vitamin supplement of the present invention can include one or more other vitamins, minerals, and/or natural ingredients. Such other vitamins, minerals, and/or natural ingredients can include, but are not limited to, one or more of the following ingredients: CoQ10, DI-Phenylalanine, Ginkgo Biloba, Hyperzine A, Inositol, Magnesium, MSM, Phosphatidiyl Serine, Potassium, White Willow, Taurine, and/or Zinc. As can be appreciated, the vitamin supplement of the present invention can include other vitamins, minerals, and/or natural ingredients. The amount of the one or more other vitamins, minerals, and/or natural ingredients that are included in the vitamin supplement of the present invention can be tailored for 1) a particular sport and/or activity, 2) a particular sex, and/or 3) a body weight range; however, this is not required. As can be appreciated, the amount of the vitamin B ingredients in the vitamin supplement of the present invention can be tailored by other or additional factors. In one non-limiting embodiment of the present invention, the vitamin supplement of the present invention includes magnesium and/or zinc. In one non-limiting aspect of this embodiment, the vitamin supplement of the present invention includes magnesium and zinc. In another and/or alternative non-limiting aspect of this embodiment, the vitamin supplement of the present invention includes 0-500 mg magnesium and/or 0-100 mg zinc. In still another and/or alternative non-limiting aspect of this embodiment, the vitamin supplement of the present invention includes 0-300 mg magnesium and/or 0-60 mg zinc. In yet another and/or alternative non-limiting aspect of this embodiment, the vitamin supplement of the present invention includes 0-250 mg magnesium and/or 0-40 mg zinc. In still yet another and/or alternative non-limiting aspect of this embodiment, the vitamin supplement of the present invention includes 50-250 mg magnesium and/or 20-40 mg zinc. In another and/or alternative non-limiting aspect of this embodiment, the vitamin supplement of the present invention includes 150-250 mg magnesium and/or 22-35 mg zinc.

In yet another and/or alternative non-limiting aspect of the present invention, the vitamin supplement of the present invention includes at least eight (8) vitamins, minerals, and natural ingredients. In one aspect of this embodiment, the vitamin supplement of the present invention includes Vitamin B1, Vitamin B2, Vitamin B3, Vitamin B5, Vitamin B6, Vitamin B12, Magnesium, and Zinc. In one embodiment of the invention, the vitamin supplement of the present invention includes at least twelve (12) vitamins, minerals, and natural ingredients. In another and/or alternative embodiment of the invention, the vitamin supplement of the present invention includes at least eighteen (18) vitamins, minerals, and natural ingredients. In one aspect of this embodiment, the vitamin supplement of the present invention includes Vitamin B1, Vitamin B2, Vitamin B3, Vitamin B5, Vitamin B6, Vitamin B12, CoQ10, DI-Phenylalanine, Ginkgo Biloba, Hyperzine A, Inositol, Magnesium, MSM, Phosphatidiyl Serine, Potassium, White Willow, Taurine, and Zinc. Several examples of the content of the vitamin supplement of the present invention are set forth as follows:

FORMULATIONS

| | Example/Ingredient | | | | | |
|---|---|---|---|---|---|---|
| | Ex. 1 | | Ex. 2 | | Ex. 3 | |
| B1 | 25-300 | mg | 50-200 | mg | 75-150 | mg |
| B2 | 25-300 | mg | 50-200 | mg | 75-150 | mg |
| B3 | 1-50 | mg | 5-30 | mg | 5-15 | mg |
| B5 | 25-300 | mg | 50-200 | mg | 75-150 | mg |
| B6 | 25-300 | mg | 50-200 | mg | 75-150 | mg |
| B12 | 100-2000 | mcg | 500-1500 | mcg | 800-1400 | mcg |
| CoQ10 | 0-20 | mg | 0-15 | mg | 2-10 | mg |
| DI- | 0-500 | mg | 0-300 | mg | 150-250 | mg |
| Phenylalanine | | | | | | |
| Ginkgo Biloba | 0-200 | mg | 0-150 | mg | 40-120 | mg |
| Hyperzine A | 0-300 | mcg | 0-200 | mcg | 50-150 | mcg |
| Inositol | 0-500 | mg | 0-350 | mg | 150-300 | mg |
| Magnesium | 0-500 | mg | 0-350 | mg | 150-350 | mg |
| MSM | 0-500 | mg | 0-350 | mg | 150-350 | mg |
| Phosphatidiyl | 0-200 | mg | 0-150 | mg | 60-120 | mg |
| Serine | | | | | | |
| Potassium | 0-300 | mg | 0-250 | mg | 50-150 | mg |
| Taurine | 0-300 | mg | 0-200 | mg | 50-150 | mg |
| White Willow | 0-500 | mg | 0-350 | mg | 150-250 | mg |
| Zinc | 0-100 | mg | 0-75 | mg | 15-45 | mg |

In still yet another and/or alternative non-limiting aspect of the present invention, the vitamin supplement of the present invention can include fillers, flavorings, emulsifiers, preservatives, etc. These other components of the vitamin supplement can be used to facilitate in the processing and/or manufacturing of the vitamin supplement, and/or to facilitate in the ingestion of the vitamin supplement by an individual.

In a further and/or alternative non-limiting aspect of the present invention, the vitamin supplement of the present invention can be formulated to be specifically directed to a particular sport such as, but not limited to the sport of golf. The specific formulation of the vitamins, mineral and natural ingredients in the vitamin supplement of the present invention can be selected to enhance the body's system to support the functions and muscle groups most critical when playing a particular sport.

In still a further and/or alternative non-limiting aspect of the present invention, the vitamin supplement of the present invention is designed so as to not require an intake build-up or a gestation period, thus the vitamin supplement can provide effective results within 0.1-2 hours of the first dosage; however, it can be appreciated that the vitamin supplement can be effective at sooner or later times. In one embodiment of the invention, the vitamin supplement is designed to provide effective results within 0.25-1 hours of the first dosage. In another and/or alternative embodiment of the invention, the vitamin supplement is designed to provide effective results within 0.5-0.75 hours of the first dosage.

In still a further and/or alternative non-limiting aspect of the present invention, the vitamin supplement of the present invention can be in a variety of forms such as, but not limited to, liquid form, tablet form, capsule form, incorporated in foods (e.g., caramel, gum, sucker, lollipop, yogurt, power bar, beverage, mints, etc.), dissolvable strips, etc. In one non-limiting embodiment of the invention is made in the form of a gelatin coated capsule which is designed to be taken about 10-60 minutes before engaging in a sport and/or other type of activity. The contents of the capsule can be liquid and/or solid. In another and/or alternative non-limiting embodiment of the invention is made in the form of a solid tablet which is designed to be taken about 10-60 minutes before engaging in a sport and/or other type of activity. In still another and/or alternative non-limiting embodiment of the invention is made in the form of a liquid which is designed to be taken about 10-60 minutes before engaging in a sport and/or other type of activity. In yet another and/or alternative non-limiting embodiment of the invention is made in the form of candy and/or gum. The gum and/or candy can be taken prior to and/or during the sport and/or other type of activity. In still yet another and/or alternative non-limiting embodiment of the invention is made in the form of a sports drink. The sports drink can be consumed prior to and/or during the sport and/or other activity. As can be appreciated, the vitamin supplement of the present invention can be in other and/or additional forms.

In yet a further and/or alternative non-limiting aspect of the present invention, the vitamin supplement of the present invention can be formulated to provide substantially continuous benefits for about 2-10 hours; however, it can be appreciated that the vitamin supplement can be effective for longer or shorter time periods. The duration of the effects of the vitamin supplement is formulated to be sufficient to last during the average period of time for a particular activity or sport. For instance, is the vitamin supplement of the present invention is formulated for a golfer, the duration of the effects of the vitamin supplement should be at least about 2-5 hours so that the average golfer playing a round of 18 hole golf will still benefit from the vitamin supplement of the present invention throughout the round of golf. In one non-limiting embodiment of the invention, the vitamin supplement is designed so as to be substantially released into the body of the user at the same time after the vitamin supplement has been consumed by the user. In another and/or alternative non-limiting embodiment of the invention, the vitamin supplement is designed so as to provide an initial released into the body of the user at the same time after the vitamin supplement has been consumed by the user, and then provide period or continuous later release of one or more vitamins so as to provide sustained dosages of one or more vitamins in the body of the user for a certain period of time.

In one non-limiting overview of the vitamin supplement of the present invention, the vitamin supplement of the present invention can be used by individual engaging in various types of sports such as, but not limited to, golf, running, bicycling, snowsports like down hill skiing, and basketball. The fundamental needs of each activity may require slight formula modifications of the vitamin supplement. The vitamin supplement is designed to provide a dosage of vitamins and minerals to a user that will enhance the user's ability to perform a physical activity. The vitamin supplement is designed to have the following features: 1) a dosage of the vitamin supplement is taken the same day of the activity, thus there is no need for a multi-day dosage to build up of the ingredients in the vitamin supplement, 2) the vitamin supplement enhances energy, focus, and/or alertness without the use of caffeine, sugars, or calories, and 3) the vitamin supplement has the ability to calm the user without sedating the user. The vitamin supplement is formulated to provide a dose of vitamin B ingredients to the user. The components and component amounts in the vitamin supplement are selected to provide enough effective ingredients into the blood stream of a 200 lb. male; however, this is not required. The dosage in the vitamin supplement is significant enough to provide substantially full effectiveness in about 30-45 minutes after initial consumption; however, longer or shorter time periods could be achieved. The dosage in the vitamin supplement is designed to last for several hours (e.g., 3-6 hours) without the user noticing a drop off in the effectiveness of the vitamin supplement. The upper limits for dosages of various components in the vitamin supplement can be set based on existing evidence in the art regarding Niacin flushing in a certain percentage of the population. The vitamin supplement can be in delivered in capsule form, rather than timed release tablet form; however, this is not required. The capsules, when used, can provide for quick effectiveness rather than a longer and slower release of a coated timed release tablet. The vitamin supplement can include many other components in addition to the vitamin B ingredients. The vitamin supplement is formulated to reduce pain and, anxiety, while increasing the focus of the user. The vitamin supplement can also contain facilitators such as, but not limited to, zinc and/or magnesium to help convert certain vitamin B vitamins into the energy and/or circulatory aids as needed.

One non-limiting object of the present invention is the provision of a vitamin supplement that reduces pain and anxiety, while increasing the focus by of the user.

Another and/or alternative non-limiting object of the present invention is the provision of a vitamin supplement that has a novel vitamin composition.

Still another and/or alternative non-limiting object of the present invention is the provision of a vitamin supplement that can be used by the user in a short time period prior to a sport and/or other activity.

Yet another and/or alternative non-limiting object of the present invention is the provision of a vitamin supplement that maintains it effectiveness several hours after being used by the user.

These and other advantages will become apparent to those skilled in the art upon the reading and following of this description.

### DETAILED DESCRIPTION OF THE INVENTION

Referring now to one or more specific embodiments of the invention for the purpose of illustrating embodiments of the invention only and not for the purpose of limiting the same, the invention comprises a mixture of the vitamin B ingredients with minerals and/or natural ingredients. The combination of the ingredients in the vitamin supplement is scientifically designed to increase energy and mental cognitive focus, as well as help the brain and central nervous system manage the stress and anxiety that is present during a round of golf, or other athletic activity. The combination of ingredients in the vitamin supplement also can help increase blood circulation throughout the body; however, this is not required. The vitamin B ingredients are believed to help to inhibit or prevent premature fatigue and to allow increased blood circulation, which will also aid the body in clearing out any and all lactic acids, thereby allowing the athlete to experience less soreness in the muscles following a round of golf or other exercise.

Two non-limiting formulations of the vitamin supplement in accordance with the present invention are set forth below:

VITAMIN SUPPLEMENT FORMULATIONS

Example/Ingredient

| | Ex. A | | Ex. B | |
|---|---|---|---|---|
| B1 | 90-130 | mg | 100 | mg |
| B2 | 90-130 | mg | 100 | mg |
| B3 | 7-13 | mg | 10 | mg |
| B5 | 90-130 | mg | 100 | mg |
| B6 | 90-130 | mg | 100 | mg |
| B12 | 1100-1300 | mcg | 1250 | mcg |
| CoQ10 | 4-8 | mg | 5 | mg |
| DI-Phenylalanine | 175-225 | mg | 200 | mg |
| Ginkgo Biloba | 60-100 | mg | 80 | mg |
| Hyperzine A | 80-130 | mcg | 100 | mcg |
| Inositol | 200-280 | mg | 250 | mg |
| Magnesium | 170-230 | mg | 200 | mg |
| MSM | 175-235 | mg | 200 | mg |
| Phosphatidiyl Serine | 60-100 | mg | 80 | mg |
| Potassium | 80-130 | mg | 100 | mg |
| Taurine | 80-125 | mg | 100 | mg |
| White Willow | 175-225 | mg | 200 | mg |
| Zinc | 25-35 | mg | 25 | mg |

Example A includes a dosage range for the components of the vitamin supplement. Example B is a very specific composition for the vitamin supplement. The composition set forth in Example B is designed for a male of average age and having a weight of about 175-225 lb. and the formulation of the vitamin supplement is selected for use in playing the game of golf. The formula of Example B is design to be used in capsule form and to be taken about 30-45 minutes prior to play a round of golf. The dosage of the vitamin supplement of Example B is selected to last the user at least about five hours. Vitamin supplements for use in other sports can have a slightly different formulation than the vitamin supplement used for play golf. Example A represents the slight adjustments to one or more of the components of the vitamin supplement for use by a male of average and a weight of about 17-225 lb. The dosage ranges in Example A are non-limiting and may be deviated from when used by males of differing age and/or weight, or when the vitamin supplement is used by females.

The believed benefits of each of the components set forth in Examples A and B are set forth below; however, it will be appreciated that one or more of the components described below may have other or additional benefits.

Vitamin B1—Helps the body form ATP, the energy or fuel on which the body runs. Nerve cells require this vitamin for normal functions under high stress.

Vitamin B2—Helps the body convert ATP into energy.

Vitamin B3—Helps the body release energy from ATP production.

Vitamin B5—Helps the body convert fat into energy and is also used to make acctylcholine, the neurotransmitter which aids the body in managing stress.

Vitamin B6—Used by the body to make the hormones serotonin and dopamine, which aid the brain with cognitive focus and ultimately provide a positive mood balance.

Vitamin B12—Used for normal cell activity during stress. It is also used for the production of S-adenofyl L-methioninc, which aids energy and mood enhancement.

CoQ10—Helps supplement cognitive focus, particularly in older males.

DI-Phenylalanine—An essential amino acid that plays an important role in mood, memory, and mental alertness.

Ginkgo Biloba—Helps focus and memory.

Hyperzine A—Helps with cognitive support.

Inositol—Helps the body cope with stress while elevating mood levels.

Magnesium—Used for activating the release of the B vitamins and is essential for helping to keep stresses muscles relaxed. Magnesium is one of the two main minerals needed by the human body for overall strength.

MSM—A natural anti-inflammatory product, and is an alternative to drugs such as ibuprofen.

Phosphatidiyl Serine—Used to facilitate a high level mental concentration.

Potassium—Used to facilitate in the body's nervous system transmission and to also facilitate in the body's transport mechanism across cell membranes.

Taurine—Helps regulate the nervous system and prevents the brain cells from hyper activity.

White Willow—A natural anti-inflammatory product, and is an alternative to drugs such as ibuprofen.

Zinc—Used to strengthen the body's immune system, especially when it is under stress. Zinc is also one of the two main minerals needed for overall body strength.

It will thus be seen that the objects set forth above, among those made apparent from the preceding description, are efficiently attained, and since certain changes may be made in the constructions set forth without departing from the spirit and scope of the invention, it is intended that all matter contained in the above description shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

We claim:

1. A vitamin supplement to temporarily enhance the abilities of a individual during a sport activity comprising a plurality of B family vitamins and one or more other vitamins, minerals, and/or natural ingredients.

2. The vitamin supplement as defined in claim 1, wherein said B family vitamins include a plurality of vitamins selected from the group consisting of Vitamin B1, Vitamin B2, Vitamin B3, Vitamin B5, Vitamin B6, Vitamin B12, or mixtures thereof.

3. The vitamin supplement as defined in claim 2, wherein said B family vitamins include at least five vitamins selected from the group consisting of Vitamin B1, Vitamin B2, Vitamin B3, Vitamin B5, Vitamin B6, Vitamin B12, or mixtures thereof.

4. The vitamin supplement as defined in claim 1, wherein said B family vitamins constitute at least about 350 mg.

5. The vitamin supplement as defined in claim 1, wherein said other vitamins, minerals, and/or natural ingredients include at least one ingredient selected from the group consisting of Magnesium, Zinc, Ginkgo Biloba, Potassium, White Willow, MSM, CoQ10, Hyperzine A, Phosphatidiyl Serine, Inositol, DL Phenylalanine, Taurine, or combinations thereof.

6. The vitamin supplement as defined in claim 3, wherein said other vitamins, minerals, and/or natural ingredients include at least one ingredient selected from the group consisting of Magnesium, Zinc, Ginkgo Biloba, Potassium, White Willow, MSM, CoQ10, Hyperzine A, Phosphatidiyl Serine, Inositol, DL Phenylalanine, Taurine, or combinations thereof.

7. The vitamin supplement as defined in claim 6, wherein said other vitamins, minerals, and/or natural ingredients include Magnesium and Zinc.

8. The vitamin supplement as defined in claim 1, including:

| | | |
|---|---|---|
| B1 | 25-300 | mg |
| B2 | 25-300 | mg |
| B3 | 1-50 | mg |
| B5 | 25-300 | mg |
| B6 | 25-300 | mg |
| B12 | 100-2000 | mcg |
| CoQ10 | 0-20 | mg |
| DI-Phenylalanine | 0-500 | mg |
| Ginkgo Biloba | 0-200 | mg |
| Hyperzine A | 0-300 | mcg |
| Inositol | 0-500 | mg |
| Magnesium | 0-500 | mg |
| MSM | 0-500 | mg |
| Phosphatidiyl Serine | 0-200 | mg |
| Potassium | 0-300 | mg |
| Taurine | 0-300 | mg |
| White Willow | 0-500 | mg |
| Zinc | 0-100 | mg |

9. The vitamin supplement as defined in claim 1, including:

| | | |
|---|---|---|
| B1 | 75-150 | mg |
| B2 | 75-150 | mg |
| B3 | 5-15 | mg |
| B5 | 75-150 | mg |
| B6 | 75-150 | mg |
| B12 | 800-1400 | mcg |
| CoQ10 | 2-10 | mg |
| DI-Phenylalanine | 150-250 | mg |
| Ginkgo Biloba | 40-120 | mg |
| Hyperzine A | 50-150 | mcg |
| Inositol | 150-300 | mg |
| Magnesium | 150-350 | mg |
| MSM | 150-350 | mg |
| Phosphatidiyl Serine | 60-120 | mg |
| Potassium | 50-150 | mg |
| Taurine | 50-150 | mg |
| White Willow | 150-250 | mg |
| Zinc | 15-45 | mg |

10. A method for enhancing the performance of a user before engaging in a sport and/or other activity comprising:
a) selecting a vitamin supplement, said vitamin supplement comprising a plurality of B family vitamins and one or more other vitamins, minerals, and/or natural ingredients; and,
b) taking said vitamin supplement at a time prior to engaging in the sport and/or other activity.

11. The method as defined in claim 10, wherein said time period is at least about 20 minutes.

12. The method as defined in claim 10, wherein said step of taking including the consumption of said vitamin supplement is capsule form.

13. The method as defined in claim 10, wherein said B family vitamins include a plurality of vitamins selected from the group consisting of Vitamin B1, Vitamin B2, Vitamin B3, Vitamin B5, Vitamin B6, Vitamin B12, or mixtures thereof.

14. The method as defined in claim 13, wherein said B family vitamins include at least five vitamins selected from the group consisting of Vitamin B1, Vitamin B2, Vitamin B3, Vitamin B5, Vitamin B6, Vitamin B12, or mixtures thereof.

15. The method as defined in claim 10, wherein said B family vitamins constitute at least about 350 mg.

16. The method as defined in claim 10, wherein said other vitamins, minerals, and/or natural ingredients include at least one ingredient selected from the group consisting of Magnesium, Zinc, Ginkgo Biloba, Potassium, White Willow, MSM, CoQ10, Hyperzine A, Phosphatidiyl Serine, Inositol, DL Phenylalanine, Taurine, or combinations thereof.

17. The method as defined in claim 15, wherein said other vitamins, minerals, and/or natural ingredients include at least one ingredient selected from the group consisting of Magnesium, Zinc, Ginkgo Biloba, Potassium, White Willow, MSM, CoQ10, Hyperzine A, Phosphatidiyl Serine, Inositol, DL Phenylalanine, Taurine, or combinations thereof.

18. The method as defined in claim 17, wherein said other vitamins, minerals, and/or natural ingredients include Magnesium and Zinc.

19. The method as defined in claim 10, including:

| | | |
|---|---|---|
| B1 | 25-300 | mg |
| B2 | 25-300 | mg |
| B3 | 1-50 | mg |
| B5 | 25-300 | mg |
| B6 | 25-300 | mg |
| B12 | 100-2000 | mcg |
| CoQ10 | 0-20 | mg |
| DI-Phenylalanine | 0-500 | mg |
| Ginkgo Biloba | 0-200 | mg |
| Hyperzine A | 0-300 | mcg |
| Inositol | 0-500 | mg |
| Magnesium | 0-500 | mg |
| MSM | 0-500 | mg |
| Phosphatidiyl Serine | 0-200 | mg |
| Potassium | 0-300 | mg |
| Taurine | 0-300 | mg |
| White Willow | 0-500 | mg |
| Zinc | 0-100 | mg |

20. The method as defined in claim 10, including:

| | | |
|---|---|---|
| B1 | 75-150 | mg |
| B2 | 75-150 | mg |
| B3 | 5-15 | mg |
| B5 | 75-150 | mg |
| B6 | 75-150 | mg |
| B12 | 800-1400 | mcg |
| CoQ10 | 2-10 | mg |
| DI-Phenylalanine | 150-250 | mg |
| Ginkgo Biloba | 40-120 | mg |
| Hyperzine A | 50-150 | mcg |
| Inositol | 150-300 | mg |
| Magnesium | 150-350 | mg |
| MSM | 150-350 | mg |
| Phosphatidiyl Serine | 60-120 | mg |
| Potassium | 50-150 | mg |
| Taurine | 50-150 | mg |
| White Willow | 150-250 | mg |
| Zinc | 15-45 | mg |
